(19) United States
(12) Patent Application Publication
(10) Pub. No.: US 2008/0265206 A1
(43) Pub. Date: Oct. 30, 2008

(54) Method for the Enzymatic Production of 5-Norbornen-2-Carboxylic Acid

(75) Inventors: Maria Kesseler, Mannheim (DE); Bernhard Haner, Fussgonheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 12/158,378

(22) PCT Filed: Dec. 11, 2006
(86) PCT No.: PCT/EP2006/069511

§ 371 Date: Jun. 20, 2008

(30) Foreign Application Priority Data
Dec. 20, 2005 (EP) 05112441.0

(51) Int. Cl. C12P 7/40; C12N 9/78; C07K 14/00; C07H 21/00; C12N 15/03; C12N 1/20; C09K 3/00

(52) U.S. Cl. 252/182.12; 435/136; 435/227; 530/350; 536/22.1; 435/320.1; 435/252.1

(57) ABSTRACT

The present invention relates to a process for the preparation of 5-norbornene-2-carboxylic acid from 5-norbornene-2-endo-carbonitrile and/or 5-norbornene-2-exo-carbonitrile. The invention relates in particular to a process which enables 5-norbornene-2-carboxylic acid to be prepared at a high substrate concentration. The invention furthermore relates to a polypeptide suitable for enzymatic conversion of 5-norbornene-2-carbonitrile to give 5-norbornene-2-carboxylic acid, in particular also with a high substrate concentration, and to a nucleic acid encoding said polypeptide, to a composition comprising 5-norbornene-2-carbonitrile to 5-norbornene-2-endo-carboxylic acid and 5-norbornene-2-exo-carboxylic acid, and to the use of said polypeptide.

The present invention relates to a process for the preparation of 5-norbornene-2-carboxylic acid from 5-norbornene-2-endo-carbonitrile and/or 5-norbornene-2-exo-carbonitrile. The invention relates in particular to a process which enables 5-norbornene-2-carboxylic acid to be prepared at a high substrate concentration. The invention furthermore relates to a polypeptide suitable for enzymatic conversion of 5-norbornene-2-carbonitrile to give 5-norbornene-2-carboxylic acid, in particular also with a high substrate concentration, and to a nucleic acid encoding said polypeptide, to a composition comprising 5-norbornene-2-carbonitrile to 5-norbornene-2-endo-carboxylic acid and 5-norbornene-2-exo-carboxylic acid, and to the use of said polypeptide.

5-Norbornene-2-carboxylic acid is used as a substrate for a multiplicity of organic syntheses and is particularly suitable for the preparation of cyclic olefin copolymers (COC), pharmaceutical intermediates, pesticides or fragrances.

Up until now, economical production of 5-norbornene-2-carboxylic acid has been possible essentially only via chemical synthesis. A particular disadvantage is the fact that the known processes result in mixtures of isomers from which the isomers must be isolated by complicated purification processes.

A process for the enzymatic preparation of 5-norbornene-2-carboxylic acid is described in Eur. J. Biochem. 182, 349-156, 1989. However, the Rhodococcus rhodochrous nitrilase described there has very low activity when converting 5-norbornene-2-carbonitrile (table 5) and is therefore not suited to enable economical production of 5-norbornene-2-carboxylic acid in a fermentative process. Moreover, the enzyme described as nitrilase in Eur. J. Biochem. 182, 349-156, 1989 was found to be a nitrile hydratase.

The invention was therefore based on the object to make available a process which could be used to prepare 5-norbornene-2-carboxylic acid in a fermentatively economical way.

The object is achieved by the process of the invention described herein and by the embodiments characterized in the claims.

The invention consequently relates to a process for enzymatic preparation of

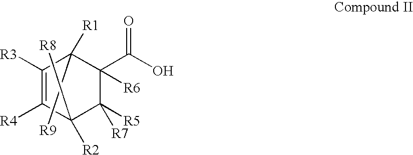

wherein
R1-R9, in each case independently of one another, may be: H, linear or branched alkyl having from one to six carbons, cycloalkyl having from two to six carbons, unsubstituted, amino-, hydroxy- or halo-substituted aryl having from 3 to 10 carbons, and wherein
R5 and R7 and also R8 and R9 may also form cycloalkyl having from 3 to 6 carbons, for example cyclopropyl, cyclobutyl, cyclopentyl or cyclohexyl; R8 and R9 and also R5 and R7 may also carry exocyclic double bonds with optional substituents; and
   R3 and R4 may form a ring (4,5,6) or may be part of an annealed aromatic compound, from

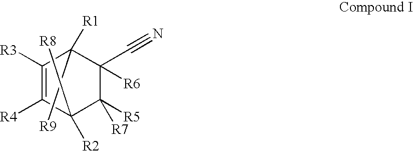

where R1 to R9 are as above,
by means of an arylacetonitrilase.

Surprisingly, it was found that it is possible to prepare compound I, in particular 5-norbornene-2-carbonitrile, to give compound II, in particular 5-norbornene-2-carboxylic acid, in an advantageous manner using arylacetonitrilases (EC 3.5.5.5). Nitrilases are enzymes which catalyze the hydrolysis of nitrites to give the corresponding carboxylic acids and ammonium ions (Faber, Biotransformations in Organic Chemistry, Springer Verlag Berlin/Heidelberg, 1992) Nitrilases have first been described in plants (Thimann and Mahadevan (1964) Arch Biochem Biophys 105:133-141) and were later found likewise in many microorganisms. Nitrilases have different substrate specificities, but may roughly be classified into three groups: nitrilases specific for aliphatic nitrites, nitrilases specific for aromatic nitrites and nitrilases specific for arylacetonitriles.

The enzymatic synthesis of chiral and achiral carboxylic acid and α-hydroxycarboxylic acids with nitrilases has been described in the prior art. Most nitrilases are very substrate-specific and can convert only a few substrates; their application is thus limited to converting only one or a few nitrites in an economically efficient manner. It is therefore advantageous to make available nitrilases capable of converting new compounds with high efficiency or under advantageous conditions.

The term “nitrilase”, as used herein, comprises any polypeptides having nitrilase activity.

The term “nitrilase activity” here means the ability to hydrolyze nitrites to give their corresponding carboxylic acids and ammonium. “Nitrilase activity” preferably means the ability of an enzyme to catalyze the addition of two molar equivalents of water to a nitrile radical, thus forming the corresponding carboxylic acid: R—CN+2H₂O [symbol] R—COOH+NH₃.

The term “nitrilase” preferably comprises enzymes of the EC classes 3.5.5.1 (nitrilases), 3.5.5.2 (ricinine nitrilases), 3.5.5.4 (cyanoalanine nitrilases), 3.5.5.5 (arylacetonitrilases), 3.5.5.6 (bromoxynil), and also 3.5.5.7 (aliphatic nitrilases). Most preference is given to arylacetonitrilases (EC 3.5.5.5).

Arylacetonitrilases (EC 3.5.5.5) are usually hardly, if at all, active with aliphatic compounds, for example propionitrile or suberonitrile and benzonitriles. It was therefore a surprise to find an arylacetonitrilase which can convert 5-norbornene-2-carbonitrile with high activity.

Preference is given in the process of the invention to compounds II.

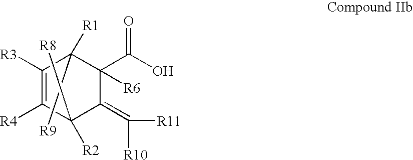

where R¹-R⁹, in each case independently of one another, may be: H, linear or branched alkyl having from one to six carbons, cycloalkyl having from two to six carbons, unsubstituted, amino-, hydroxy- or halo-substituted aryl having from 3 to 10 carbons, and wherein
R⁵ and R⁷ and also R⁰ and R⁹ may also form cycloalkyl having from 3 to 6 carbons, for example cyclopropyl, cyclobutyl, cyclopentyl or cyclohexyl;
R⁸ and R⁹ and also R⁵ and R⁷ may also carry exocyclic double bonds with optional substituents, as shown in compound IIb with R⁵, R⁷, R^{10,11}, for example, in each case independently of one another being H, alkyl or aryl having from one to six carbons; and
   R³ and R⁴ may form a ring (4,5,6) or may be part of an annealed aromatic compound,
   with compound I being:

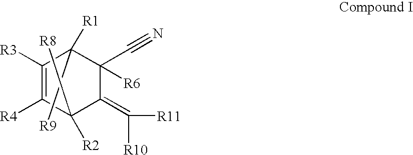

where R1 to R11 are as above.

According to the invention, the enzymes used, having the activity of the invention, may be used for converting compound I into II in the process of the invention as processed microorganisms or cells, for example as disrupted, free or immobilized enzymes, microorganisms or cells, or as partially or completely purified enzyme preparations, for example in a free or immobilized form.

Consequently, it is also possible to use in the process of the invention growing cells which comprise the nucleic acids, nucleic acid constructs or vectors of the invention. It is also possible to use resting or disrupted cells. Disrupted cells mean, for example, cells which have been made permeable, for example by treatment with solvents, or cells which have been disrupted by enzymatic treatment, for example lyzed, by mechanical treatment (e.g. French press or ultrasound) or by another method. The crude extracts obtained in this way are advantageously suitable for the process of the invention. Purified or partially purified enzymes may also be used for the process. Likewise suitable are immobilized microorganisms or enzymes which may be applied advantageously in the reaction.

If free organisms or enzymes are used for the process of the invention, then these are conveniently removed, for example by filtration or centrifugation, prior to the extraction.

A microorganism according to the present invention may be cultured or propagated in a medium which allows this microorganism to grow. The medium may be of synthetic or natural origin. Various media for microorganisms are known. For growth of the microorganisms, the medium comprises a carbon source, a nitrogen source, inorganic salts and optionally small amounts of vitamins and/or trace elements.

Examples of preferred carbon sources are polyols such as, for example, glycerol, sugars such as, for example, mono-, di- or polysaccharides (e.g. glucose, fructose, manose, xylolose, galactose, ribose, sorbose, ribulose, lactose, maltose, succose, rafinose, starch or cellulose), complex sugar sources (e.g. molasses), sugar phosphates (e.g. fructose-1-ex-biphosphate), sugar alcohols (e.g. mannitol), alcohols (e.g. methanol or ethanol), carboxylic acids (e.g. soybean oil or linseed oil), amino acids or amino acid mixtures (e.g. casamino acids, Difco) or particular amino acids (e.g. glycine, asparagine) or amino saccharides, it being possible for the latter to be used also as nitrogen sources. Particular preference is given to glucose and polyols, in particular glycerol.

Preferred nitrogen sources are organic and inorganic nitrogen compounds or materials which comprise these compounds. Examples of good nitrogen sources are ammonium salts (e.g. NH₄Cl or (NH₄)₂SO₄), nitrates, urea, and complex nitrogen sources such as, for example, yeast lysates, soybean meal, wheat gluten, yeast extract, peptone, meat extract, casein hydrolyzates, yeast or potato protein, it being possible for the latter to serve also as carbon sources.

Examples of inorganic salts comprise calcium, magnesium, sodium, cobalt, manganese, potassium, zinc, copper and iron salt. Corresponding anions which are particularly preferred are chloride, sulfate, sulfite and phosphate ions. An important factor for good productivity is the control of the Fe2+- or Fe3+-ion concentration in the medium.

The medium may optionally and additionally comprise growth factors such as, for example, vitamins or growth enhancers such as biotin, 2-keto-1-gulonic acid, ascorbic acid, thiamine, folic acid, amino acids, carboxylic acids or substances such as, for example, DTT.

The fermentation and growth conditions are selected so that a high yield of the desired product can be achieved (e.g. high nitrilase activity, in particular high arylacetonitrilase activity). Preferred fermentation conditions are between 15° C. and 40° C., preferably 25° C. to 37° C. The pH is preferably regulated in the range from pH 3 to 9, even more preferably between pH 5 and 8. The duration of the fermentation is generally between a few hours and a few days, preferably between 8 hours and 21 days, more preferably 4 hours and 14 days. Processes for optimization of medium and fermentation conditions are known in the prior art (Applied Microbiol Physiology, A practical approach 1997, pages 53 to 73).

In one embodiment, the process of the invention is carried out so that enzymatic conversion of compound I into compound II is carried out by way of incubation with a polypeptide or a medium comprising a polypeptide and wherein said polypeptide is encoded by a nucleic acid molecule comprising a nucleic acid molecule selected from the group consisting of:
(a) nucleic acid molecule which encodes a polypeptide depicted in SEQ ID NO: 2 or 4;
(b) nucleic acid molecule which comprises at least the polynucleotide of the coding sequence according to SEQ ID NO: 1 or 3;
(c) nucleic acid molecule whose sequence, owing to the degeneracy of the genetic code, may be derived from a polypeptide sequence encoded by a nucleic acid molecule according to (a) or (b);
(d) nucleic acid molecule which encodes a polypeptide whose sequence is at least 60% identical to the amino acid sequence of the polypeptide encoded by the nucleic acid molecule according to (a) or (b);
(e) nucleic acid molecule which encodes a polypeptide derived from an arylaceto-nitrilase polypeptide in which up to 25% of the amino acid residues have been modified by deletion, insertion, substitution or a combination thereof compared to SEQ ID NO: 2 and which still retains at least 30% of the enzymatic activity of SEQ ID NO: 2; and
(f) nucleic acid molecule which encodes a fragment or an epitope of an arylaceto-nitrilase encoded by any of the nucleic acid molecules according to (a) to (c);
   or comprising a complementary sequence thereof;
   and, optionally, the product formed is isolated.

Preferred enzymes having the activity of the invention comprise an amino acid sequence according to SEQ ID NO: 2 or 4.

The nitrilase of the invention hydrolyzes very well phenylacetonitrile>phenylpropionitrile>mandelonitrile (moderate enantioselectivity) and is hardly or not at all active with aliphatic compounds (e.g. propionitrile, suberonitrile) or benzonitriles. Activity with norbornene nitrites, in particular, is therefore a surprise.

Advantageous is moreover the enormous stability and productivity of the enzyme of the invention under reactor condition and the easy handling, since a wide temperature and pH range is available and the enzyme has a high tolerance to nitrile, i.e. it is not necessary to measure out nitrile.

The invention likewise comprises “functional equivalents” of the specifically disclosed enzymes having the activity of the invention and the use of these equivalents in the processes of the invention.

“Functional equivalents” or analogs of the specifically disclosed enzymes are, for the purposes of the present invention, polypeptides which differ therefrom and which furthermore possess the desired biological activity such as, for example, substrate specificity. Thus, for example, “functional equivalents” mean enzymes which convert from compound I to compound II and which have at least 50%, preferably 60%, particularly preferably 75%, very particularly preferably 90% or more, of the activity of an enzyme having the amino acid sequence listed under SEQ ID NO: 2. Moreover, functional equivalents are preferably stable at temperatures from 0° C. to 70° C. and advantageously possess a pH optimum between pH 5 and 8 and a temperature optimum in the range from 10° C. to 50° C.

“Functional equivalents” mean, according to the invention, in particular also mutants which have in at least one sequence position of the abovementioned amino acid sequences an amino acid other than the specifically mentioned one but which nevertheless possess one of the abovementioned biological activities. “Functional equivalents” thus comprise the mutants obtainable by one or more amino acid additions, substitutions, deletions and/or inversions, it being possible for said modifications to occur in any sequence position, as long as they result in a mutant having the property profile of the invention. Functional equivalence in particular also exists, if the reactivity patterns between the mutant and the unmodified polypeptide correspond qualitatively, i.e., for example, the same substrates are converted at different rates.

Examples of suitable amino acid substitutions can be found in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” mean, according to the invention, in particular also mutants which have in at least one sequence position of the abovementioned amino acid sequences an amino acid other than the specifically mentioned one but which nevertheless possess one of the abovementioned biological activities. “Functional equivalents” thus comprise the mutants obtainable by one or more amino acid additions, substitutions, deletions and/or inversions, it being possible for said modifications to occur in any sequence position, as long as they result in a mutant having the property profile of the invention. Functional equivalence in particular also exists, if the reactivity patterns between the mutant and the unmodified polypeptide correspond qualitatively, i.e., for example, the same substrates are converted at different rates, with the rate being not less than 30% of that of the unmodified polypeptide, preferably more than 100%, in particular more than 150%, particularly preferably a rate increased by a factor of 2, 5 or 10.

“Functional equivalents” in the above sense are also “precursors” of the described polypeptides, and “functional derivatives” and “salts” of the polypeptides.

“Precursors” are in this connection natural or synthetic precursors of the polypeptides with or without the desired biological activity.

The term “salts” means both salts of carboxyl groups and acid addition salts of amino groups of the protein molecules of the invention. Salts of carboxyl groups can be prepared in a manner known per se and comprise inorganic salts such as, for example, sodium, calcium, ammonium, iron and zinc salts, and salts with organic bases such as, for example, amines, such as triethanolamine, arginine, lysine, piperidine and the like. The invention likewise relates to acid addition salts such as, for example, salts with mineral acids such as hydrochloric acid or sulfuric acid and salts with organic acids such as acetic acid and oxalic acid.

“Functional derivatives” of polypeptides of the invention can likewise be prepared on functional amino acid side groups or on the N- or C-terminal end thereof by means of known techniques. Such derivatives comprise for example aliphatic esters of carboxylic acid groups, amides of carboxylic acid groups, obtainable by reaction with ammonia or with a primary or secondary amine; N-acyl derivatives of free amino groups prepared by reaction with acyl groups; or O-acyl derivatives of free hydroxy groups prepared by reaction with acyl groups.

“Functional equivalents” naturally also comprise polypeptides which are obtainable from other organisms, and naturally occurring variants. It is possible for example to establish ranges of homologous sequence regions by comparison of sequences, and to ascertain equivalent enzymes based on the specific requirements of the invention.

“Functional equivalents” likewise comprise fragments, preferably single domains or sequence motifs, of the polypeptides of the invention, which have, for example, the desired biological function.

“Functional equivalents” are additionally fusion proteins which comprise one of the abovementioned polypeptide sequences or functional equivalents derived therefrom and at least one further, heterologous sequence which is functionally different therefrom and is in functional N- or C-terminal linkage (i.e. with negligible mutual functional impairment of the parts of the fusion protein). Nonlimiting examples of such heterologous sequences are, for example, signal peptides or enzymes.

“Functional equivalents” also included in the invention are homologs of the specifically disclosed proteins. These have a homology of at least 60%, preferably at least 75%, in particular at least 85%, such as, for example, 90%, 95% or 99%, with one of the specifically disclosed amino acid sequences calculated by the algorithm of Pearson and Lipman, Proc. Natl. Acad, Sci. (USA) 85(8), 1988, 2444-2448. A percentage homology of a homologous polypeptide of the invention means in particular percentage identity of the amino acid residues based on the total length of one of the amino acid sequences specifically described herein.

In the case of possible protein glycosylation, “functional equivalents” of the invention comprise proteins of the type defined above in deglycosylated or glycosylated form, and modified forms obtainable by altering the glycosylation pattern.

Homologs of the proteins or polypeptides of the invention can be generated by mutagenesis, e.g. by point mutation or truncation of the protein.

Homologs of the proteins of the invention can be identified by screening combinatorial libraries of mutants, such as, for example, truncation mutants. For example, a variegated library of protein variants can be generated by combinatorial mutagenesis at the nucleic acid level, such as, for example, by enzymatic ligation of a mixture of synthetic oligonucleotides. There is a large number of methods which can be used to prepare libraries of potential homologs from a degenerate oligonucleotide sequence. Chemical synthesis of a degenerate gene sequence can be carried out in an automatic DNA synthesizer, and the synthetic gene can then be ligated into a suitable expression vector. The use of a degenerate set of genes makes it possible to provide all the sequences which encode the desired set of potential protein sequences in one mixture. Methods for synthesizing degenerate oligonucleotides are known to the skilled worker (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

Several techniques are known in the art for screening gene products in combinatorial libraries which have been prepared by point mutations or truncation, and for screening cDNA libraries for gene products having a selected property. These techniques can be adapted to the rapid screening of gene libraries which have been generated by combinatorial mutagenesis of homologs of the invention. The most commonly used techniques for screening large gene libraries, which are subject to high-throughput analysis, comprise the cloning of the gene library into replicable expression vectors, transformation of suitable cells with the resulting vector library and expression of the combinatorial genes under conditions under which detection of the desired activity facilitates isolation of the vector which encodes the gene whose product has been detected. Recursive ensemble mutagenesis (REM), a technique which increases the frequency of functional mutants in the libraries, can be used in combination with the screening tests to identify homologs (Arkin and Yourvan (1992) PNAS 89:7811-7815; Delgrave et al. (1993) Protein Engineering 6(3):327-331).

In one embodiment the process of the invention is carried out at a reaction temperature from 5 to 75° C. The reaction temperature is preferably ambient or room temperature or higher, for example 30° C. or higher, but lower than 70° C., preferably 60° C., 50° C. or lower. In a preferred embodiment, the reaction temperature for preparing xNon is approximately from 35 to 45° C., for example 40° C. In a preferred embodiment, the reaction temperature for preparing eNon is between ambient temperature and 50° C.

Compound I may be both a mixture of enantiomers, for example R,S or end/exo enantiomers, and enantiomerically pure, i.e. comprise mainly one enantiomer. In one embodiment, the process of the invention involves converting an enantiomerically pure substrate.

In the process of the invention, isomerically pure, enantiomerically pure or chiral products or optically active compounds mean enantiomers which show enrichment of one enantiomer. The process preferably achieves enantiomeric purities of at least 70% ee, preferably of at least 80% ee, particularly preferably of at least 90% ee, very particularly preferably at least 98% ee, even more preferably 99% ee, and most preferably of at least 99.5% ee.

In one embodiment, the process of the invention involves hydrolyzing R-5-norbornene-2-endo-carbonitrile, S-5-norbornene-2-endo-carbonitrile, R-5-norbornene-2-exo-carbonitrile, and/or S-5-norbornene-2-exo-carbonitrile to give the corresponding S-5-norbornene-2-exo-carboxylic acid, S-5-norbornene-2-endo-carboxylic acid, R-5-norbornene-2-exo-carboxylic acid and R-5-norbornene-2-endo-carboxylic acid, respectively.

In a further embodiment, compound I equals R-5-norbornene-2-endo-carbonitrile and S-5-norbornene-2-endo-carbonitrile or R-5-norbornene-2-exo-carbonitrile and S-5-norbornene-2-exo-carbonitrile.

In another embodiment, compound I equals R-5-norbornene-2-endo-carbonitrile or S-5-norbornene-2-endo-carbonitrile or R-5-norbornene-2-exo-carbonitrile or S-5-norbornene-2-exo-carbonitrile.

Consequently, the invention also relates to a process in which an enantiomerically pure product is obtained.

In one embodiment, the invention relates to a process in which at a substrate concentration is at least 20 mM, preferably 50 mM, 70 mM, 100 mM, 150 mM, 200 mM, 250 mM, 300 mM, 400 mM, 500 mM, 700 mM, 1000 mM, 2000 mM, or more and wherein at least 50%, preferably 60%, 65%, 70%, 75%, 80%, 85%, 90%, 95%, or more of the substrate, i.e. compound I, in particular R-5-norbornene-2-endo-carbonitrile, S-5-norbornene-2-endo-carbonitrile, R-5-norbornene-2-exo-carbonitrile, and/or S-5-norbornene-2-exo-carbonitrile, are converted to give compound II.

In one embodiment, the substrate used is a mixture of isomers, in particular a mixture of enantiomers, of compound I, with one isomer, in particular one enantiomer of compound II, being enriched in the product. Preference is given to using in the process of the invention an endow and exo-enantiomer of compound I with the endo- or exo-enantiomer of compound II being enriched. Particular preference is given to hydrolyzing in the process of the invention for enrichment a mixture of R-5-norbornene-2-endo-carbonitrile and/or S-5-norbornene-2-endo-carbonitrile and R-5-norbornene-2-exo-carbonitrile and/or S-5-norbornene-2-exo-carbonitrile to give the corresponding S-5-norbornene-2-exo-carboxylic acid and/or R-5-norbornene-2-exo-carboxylic acid and R-5-norbornene-2-endo-carboxylic acid and/or S-5-norbornene-2-endo-carboxylic acid with preferably the endo-enantiomers of norbornene acid being enriched.

The pH in the process of the invention is advantageously maintained between pH 6 and 10, preferably between pH 7 and 9, particularly preferably between pH 7.5 and 8.5.

The product prepared in the process of the invention, for example R- and/or S-5-norbornene-2-exo-carboxylic acid and/or R- and/or S-5-norbornene-2-endo-carboxylic acid, can advantageously be isolated from the aqueous reaction solution by extraction or distillation. To increase the yield, the extraction may be repeated several times. Examples of suitable extractants are solvents such as toluene, methylene chloride, butyl acetate, diisopropyl ether, benzene, MTBE or ethyl acetate, without being limited thereto.

After concentration of the organic phase, the products can usually be obtained in good chemical purities, i.e. greater than 80%, preferably 85%, 90%, 95%, 98% or more, chemical purity. After extraction, the organic phase containing the product can, however, also be only partly concentrated, and the product can be crystallized out. For this purpose, the solution is advantageously cooled to a temperature of from 0° C. to 10° C. Crystallization is also possible directly from the organic solution or from an aqueous solution. The crystallized product can be taken up again in the same or in a different solvent for recrystallization and be crystallized again.

It is possible, by carrying out the subsequent optional crystallization preferably at least once, to increase the enantiomeric purity of the product further if necessary.

With the types of workup mentioned, the product of the process of the invention can be isolated in yields of from 60 to 100%, preferably from 80 to 100%, particularly preferably from 90 to 100%, based on the substrate employed for the reaction, such as R-5-norbornene-2-endo-carbonitrile, R-5-norbornene-2-exo-carbonitrile S-5-norbornene-2-endo-carbonitrile, and/or S-5-norbornene-2-exo-carbonitrile, for example. The isolated product is distinguished by a high chemical purity of >90%, preferably >95%, particularly preferably >98%. Furthermore, the products have a high enantiomeric purity which can advantageously be further increased, if necessary, by said crystallization.

The process of the invention can be carried out batchwise, semibatchwise or continuously.

The process may advantageously be carried out in bioreactors as described, for example, in Biotechnology, volume 3, 2nd edition, Rehm et al Eds., (1993), in particular Chapter II.

In one embodiment, the invention also relates to a polypeptide which is suitable for enzymatically hydrolyzing compound I to give compound II. Said polypeptide preferably encodes a nitrilase, in particular an arylacetonitrilase.

In one embodiment, the polypeptide is encoded by a nucleic acid molecule comprising a nucleic acid molecule selected from the group consisting of:
(a) nucleic acid molecule which encodes a polypeptide depicted in SEQ ID NO: 2 or 4.
(b) nucleic acid molecule which comprises at least the polynucleotide of the coding sequence according to SEQ ID NO: 1 or 3;
(c) nucleic acid molecule whose sequence, owing to the degeneracy of the genetic code, may be derived from a polypeptide sequence encoded by a nucleic acid molecule according to (a) or (b);
(d) nucleic acid molecule which encodes a polypeptide whose sequence is at least 60% identical to the amino acid sequence of the polypeptide encoded by the nucleic acid molecule according to (a) or (b);
(e) nucleic acid molecule which encodes a polypeptide derived from an arylaceto-nitrilase polypeptide in which up to 15% of the amino acid residues have been modified by deletion, insertion substitution or a combination thereof compared to SEQ ID NO: 2 or 4 and which still retains at least 30% of the enzymatic activity of SEQ ID NO: 2 or 4; and
(f) nucleic acid molecule which encodes a fragment or an epitope of an arylacetonitrilase encoded by any of the nucleic acid molecules according to (a) to (c);
   or comprising a complementary sequence thereof.

In one embodiment, the polypeptide does not have the sequence according to SEQ ID NO: 2 and/or 4. In one embodiment, the polypeptide neither has the sequence of the nitrilase mentioned in Eur. J. Biochem. 182, 349-156, 1989. In one embodiment, the polypeptide neither has the sequence of the database entry AY885240.

In one embodiment, the polypeptide of the invention has the property of producing a high percentage of compound II, in particular norbornene acid, even at a high substrate concentration, i.e. at a high concentration of compound I in the medium. The polypeptide is preferably capable of converting, at a 5-norbornene-2-endo-carbonitrile concentration of 20 mM, preferably 50 mM, 70 mM, 100 mM, 150 mM, 200 mM, 250 mM, 300 mM, 400 mM, 500 mM, 700 mM, 1000 mM, 2000 mM, or more, at least 50%, preferably 60%, 65%, 70%, 75%, 80%, 85%, 90%, 95%, or more of the substrate to give compound II, said substrate, i.e. compound I, being in particular R-5-norbornene-2-endo-carbonitrile, S-5-norbornene-2-endo-carbonitrile, R-5-norbornene-2-exo-carbonitrile, and/or S-5-norbornene-2-exo-carbonitrile. Particular preference is given to the polypeptide converting at least 65% of the substrate at a substrate concentration of at least 150 mM at 40° C. within 24 h.

Consequently, the invention also relates to a nucleic acid molecule which encodes the polypeptide of the invention. The present invention furthermore relates to a nucleic acid molecule comprising a polynucleotide encoding a polypeptide of the invention. In one embodiment, the nucleic acid molecule does not have the sequence of SEQ ID NO: 1. In one embodiment, the nucleic acid molecule does not encode the nitrilase of Eur. J. Biochem. 182, 349-156, 1989. In one embodiment, the nucleic acid molecule does also not have the sequence of the database entry AY885240.

The invention relates in particular to nucleic acid sequences (single- and double-stranded DNA and RNA sequences such as, for example, cDNA and mRNA) which code for an enzyme having activity according to the invention or which can be employed in the process of the invention. Preference is given to nucleic acid sequences which code, for example, for amino acid sequences according to SEQ ID NO: 2 or 4 or characteristic partial sequences thereof or which comprise nucleic acid sequences according to SEQ ID NO: 1 or 3 or characteristic partial sequences thereof.

All nucleic acid sequences mentioned herein can be prepared in a manner known per se by chemical synthesis from the nucleotide building blocks, for example by fragment condensation of individual overlapping, complementary nucleic acid building blocks of the double helix. The chemical synthesis of oligonucleotides can take place, for example, in the known manner by the phosphoamidite method (Voet, Voet, 2nd edition, Wiley Press New York, pages 896-897). Addition of synthetic oligonucleotides and filling gaps with the aid of the Klenow fragment of DNA polymerase and ligation reactions, and also general cloning methods, are described in Sambrook et al. (1989), Molecular Cloning: A laboratory manual, Cold Spring Harbor Laboratory Press.

The invention also relates to nucleic acid sequences (single- and double-stranded DNA and RNA sequences such as, for example, cDNA and mRNA) coding for any of the above polypeptides and their functional equivalents which are accessible using, for example, artificial nucleotide analogs.

In one embodiment, the nucleic acid sequence of the invention differs by at least one base from the sequence of SE ID NO: 1 or 3. In one embodiment, the nucleic acid molecule does also not have the sequence of the nitrilase mentioned in Eur. J. Biochem. 182, 349-156, 1989. In one embodiment, the nucleic acid molecule neither has the sequence of the database entry AY885240.

The invention relates to both isolated nucleic acid molecules coding for polypeptides or proteins of the invention or biologically active sections thereof and nucleic acid fragments which may be used, for example, for use as hybridization probes or primers for identifying or amplifying coding nucleic acids of the invention.

The nucleic acid molecules of the invention may moreover comprise untranslated sequences from the 3′ and/or 5′ end of the coding gene region.

The invention furthermore comprises the nucleic acid molecules complementary to the specifically described nucleotide sequences or a section thereof.

The nucleotide sequences of the invention make it possible to generate probes and primers which can be used for identifying and/or cloning homologous sequences in other cell types and organisms. Probes and primers of this kind usually comprise a nucleotide sequence region which hybridizes, under “stringent” conditions (see below), to at least about 12, preferably at least about 25, such as, for example, about 40, 50 or 75, consecutive nucleotides of a sense strand of a nucleic acid sequence of the invention or of a corresponding antisense strand.

An “isolated” nucleic acid molecule is removed from other nucleic acid molecules which are present in the natural source of the nucleic acid and may moreover be essentially free of other cellular material or culture medium when it is prepared by means of recombinant techniques or free of chemical precursors or other chemicals when it is synthesized chemically.

A nucleic acid molecule of the invention may be isolated by means of standard molecular-biological techniques and the sequence information which is provided according to the invention. For example, cDNA may be isolated from a suitable cDNA library by using one of the specifically disclosed complete sequences or a section thereof as hybridization probe and using standard hybridization techniques (as described, for example, in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, N.Y., 1989). In addition, a nucleic acid molecule comprising any of the disclosed sequences or a section thereof can be isolated by polymerase chain reaction, the oligonucleotide primers which have been constructed on the basis of this sequence being used. The nucleic acid amplified in this way may be cloned into a suitable vector and characterized by DNA sequence analysis. The oligonucleotides of the invention may also be prepared by standard synthesis processes using, for example, an automatic DNA synthesizer.

The nucleic acid sequences of the invention can be identified and isolated in principle from any organisms. Advantageously, the nucleic acid sequences of the invention or the homologs thereof can be isolated from fungi, yeasts, archeae or bacteria. Bacteria which may be mentioned are Gram-negative and Gram-positive bacteria. The nucleic acids of the invention are preferably isolated from Gram-negative bacteria, advantageously from α-proteobacteria, β-proteobacteria or γ-proteobacteria, particularly preferably from bacteria of the orders Burkholderiales, Hydrogenophilales, Methylophilales, Neisseriales, Nitrosomonadales, Procabacteriales or Rhodocyclales. Very particularly preferably from bacteria of the family Rhodocyclaceae.

Particular preference is given to using arylacetonitrilases from Pseudomonas spec.

Nucleic acid sequences of the invention can, for example, be isolated from other organisms by using customary hybridization processes or the PCR technique, for example by way of genomic or cDNA libraries. These DNA sequences hybridize with the sequences of the invention under standard conditions. Use is advantageously made, for the hybridization, of short oligonucleotides of the conserved regions, for example from the active site, which conserved regions may be identified in a manner known to the skilled worker by way of comparisons with a nitrilase of the invention, in particular arylacetonitrilases. However, it is also possible to use longer fragments of the nucleic acids of the invention or the complete sequences for the hybridization. Said standard conditions vary depending on the nucleic acid employed (oligonucleotide, longer fragment or complete sequence) or depending on which nucleic acid type, DNA or RNA, is used for the hybridization. Thus, for example, the melting temperatures for DNA:DNA hybrids are approx. 10° C. lower than those for DNA:RNA hybrids of the same length.

The invention also relates to derivatives of the specifically disclosed or derivable nucleic acid sequences.

Thus, further nucleic acid sequences of the invention may be derived from SEQ ID NO: 1 or 3 and differ therefrom by the addition, substitution, insertion or deletion of single or two or more nucleotides but still code for polypeptides having the desired property profile.

The invention also comprises those nucleic acid sequences which comprise “silent” mutations or have been altered, as compared with a specifically mentioned sequence, according to the codon usage of a specific source organism or host organism, as well as naturally occurring variants thereof, such as splice variants or allele variants, for example.

The invention also relates to sequences obtainable by way of conservative nucleotide substitutions (i.e. the amino acid in question is replaced with an amino acid of the same charge, size, polarity and/or solubility).

The invention also relates to the molecules which are derived from the specifically disclosed nucleic acids by way of sequence polymorphisms. These genetic polymorphisms can exist between individuals within a population as a result of natural variation. These natural variations usually give rise to a variance of from 1 to 5% in the nucleotide sequence of a gene.

Derivatives of a nucleic acid sequence of the invention mean, for example, allele variants which have at least 50% homology at the deduced amino acid level, preferably at least 75% homology, very particularly preferably at least 80, 85, 90, 93, 95, 98 or 99%, homology over the entire sequence region (regarding homology at the amino acid level, the reader is referred to the above comments on the polypeptides). The homologies may be advantageously higher across subregions of said sequences.

Derivatives furthermore also mean homologs of the nucleic acid sequences of the invention, for example fungal or bacterial homologs, truncated sequences, single-stranded DNA or RNA of the coding and noncoding DNA sequence. Thus, for example at the DNA level, have a homology of at least 50%, preferably of 75% or more, particularly preferably of 80%, very particularly preferably of 90%, most preferably 95%, in particular 98%, or more, across the entire DNA region indicated.

According to the invention, “homolog” or “substantial sequence homology” generally means that the nucleic acid sequence of a DNA molecule or the amino acid sequence of a protein is at least 40%, preferably at least 50%, further preferably at least 60%, likewise preferably at least 70%, particularly preferably at least 90%, especially preferably at least 95% and most preferably at least 98%, identical to the nucleic acid or amino acid sequences of the arylacetonitrilases, in particular to SEQ ID NO: 1, 2, 3 or 4 or the functionally equivalent parts thereof. The homology is preferably determined over the entire length of the sequence of the arylacetonitrilases, in particular to SEQ ID NO:1, 2, 3 or 4.

“Identity between two proteins” means the identity of the amino acids across a particular protein region, preferably over the entire length of the protein, in particular the identity calculated by way of comparison with the aid of the Laser gene software from DNA Star Inc., Madison, Wis. (USA), applying the CLUSTAL method (Higgins et al., 1909), Comput. Appl. Biosci., 5 (2), 151). Homologies may likewise be calculated with the aid of the Laser gene software from DNA Star Inc., Madison, Wis. (USA), applying the CLUSTAL method (Higgins et al., 1989), Comput. Appl. Biosci., 5 (2), 151). The sequence comparisons may be carried out using the pre-set parameters of the page http://www.ebi.ac.uk/clustalw/ last updated: Oct. 17, 2005 11:27:35, with the following programs in the FTP DIRECTORY:

ftp://ftp.ebi.ac.uk/pub/software/unix/clustalw/

| | |
|---|---|
| ParClustal0.1.tar.gz | [Nov 28 2001] 823975 |
| ParClustal0.2.tar.gz | [Jun 27 2002] 2652452 |
| README | [Jun 13 2003] 673 |
| clustalw1.8.UNIX.tar.gz | [Jul 4 1999] 4725425 |
| clustalw1.8.mp.tar.gz | [May 2 2000] 174859 |
| clustalw1.81.UNIX.tar.gz | [Jun 7 2000] 555655 |
| clustalw1.82.UNIX.tar.gz | [Feb 6 2001] 606683 |
| clustalw1.82.mac-osx.tar.gz | [Oct 15 2002] 669021 |
| clustalw1.83.UNIX.tar.gz | [Jan 30 2003] 166863 |

as depicted in FIG. 2.

Thus, the homology is preferably calculated over the entire region of the amino acid or nucleic acid sequence. Apart from the abovementioned programs, there are still other programs for the comparison of various sequences available to the skilled worker, which programs are based on various algorithms, with the algorithms by Meedleman and Wunsch or Smith and Waterman giving particularly reliable results. Sequence comparisons may also be carried out using the Pile Aupa program (J. Mol. Evolution. (1987), 25, 351-360; Higgins et al., (1989) Cabgos, 5, 151-153), for example, or the Gap and Best Fit programs (Needleman and Wunsch, (1970), J. Mol. Biol., 48, 443-453 and Smith and Waterman (1981), Adv., Appl. Math., 2, 482-489) which are part of the GCG software package of Genetics Computer Group (575 Science Drive, Madison, Wis., USA 53711). In a further, particularly preferred embodiment of the present invention, the homology over the cDNA full length sequence is determined using the Gap program. In a further, particularly preferred embodiment of the present invention, the homology over the entire genomic sequence is determined using the Gap program, In a very particularly preferred embodiment of the present invention, the homology over the coding full length sequence is determined using the Gap program. Moreover, derivatives mean fusions with promoters, for example. The promoters which are located upstream of the nucleotide sequences indicated may have been altered by one or more nucleotide replacements, insertions, inversions and/or deletions without, however, the functionality and efficacy of the promoters being impaired. Furthermore, the efficacy of said promoters may be increased by altering their sequence or the promoters may be completely replaced with more active promoters, including those from organisms of other species.

Derivatives also mean variants whose nucleotide sequence in the region from −1 to −1000 bases upstream of the start codon or from 0 to 1000 bases downstream of the stop codon has been altered so as to alter, preferably increase, gene expression and/or protein expression.

The invention furthermore comprises nucleic acid sequences which hybridize with coding sequences mentioned above under “stringent conditions”. The term “stringent conditions” therefore refers to conditions under which a nucleic acid sequence preferentially binds to a target sequence but does not bind to other sequences or binds thereto at least in a substantially reduced manner.

These polynucleotides can be found by screening genomic or cDNA libraries and, if appropriate, amplified therefrom by means of PCR using suitable primers and then isolated using suitable probes, for example. In addition, polynucleotides of the invention may also be synthesized chemically. This property means the ability of a polynucleotide or oligonucleotide to bind to a virtually complementary sequence under stringent conditions while, under these conditions, unspecific bonds between noncomplementary partners are not formed. For this purpose, the sequences should be 70-100%, preferably 90-100%, complementary. The property of complementary sequences of being able to bind specifically to one another is utilized, for example, in the Northern or Southern blot technique or for primer binding in PCR or RT-PCR. Oligonucleotides of at least 30 base pairs in length are usually used for this purpose.

Depending on the nucleic acid, standard conditions mean, for example, temperatures between 42 and 58° C. in an aqueous buffer solution having a concentration of between 0.1 to 5×SSC (1×SSC=0.15 M NaCl, 15 mM sodium citrate, pH 7.2) or additionally in the presence of 50% formamide, such as, for example, 42° C. in 5×SSC, 50% formamide. Advantageously, the hybridization conditions for DNA:DNA hybrids are 0.1×SSC and temperatures between about 20° C. to 45° C., preferably between about 30° C. to 45° C. For DNA:RNA hybrids, the hybridization conditions are advantageously 0.1×SSC and temperatures between about 30° C. to 55° C., preferably between about 45° C. to 55° C. The temperatures indicated for the hybridization are melting temperature values which have been calculated by way of example for a nucleic acid having a length of approx. 100 nucleotide and a G+C content of 50% in the absence of formamide. The experimental conditions for the DNA hybridization are described in specialist textbooks of genetics, such as, for example, Sambrook et al., “Molecular Cloning”, Cold Spring Harbor Laboratory, 1989, and can be calculated using formulae known to the skilled worker, for example as a function of the length of the nucleic acids, the type of hybrids or the G+C content. The skilled worker can obtain further information with regard to hybridization from the following textbooks: Ausubel et al. (eds), 1985, Current Protocols in Molecular Biology, John Wiley & Sons, New York, Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

In the Northern blot technique, for example, stringent conditions mean the use of a washing solution of 50-70° C., preferably 60-65° C., for example 0.1×SSC buffer containing 0.1% SDS (20×SSC: 3M NaCl, 0.3M sodium citrate, pH 7.0), for eluting unspecifically hybridized cDNA probes or oligonucleotides. As mentioned above, the only nucleic acids to remain bound to one another here are those which are highly complementary. The establishment of stringent conditions is known to the skilled worker and is described, for example, in Ausubel et al., Current Protocols in Molecular Biology, John Wiley & Sons, N.Y. (1989), 6.3.1-6.3.6.

The term “complementarity” describes the ability of a nucleic acid molecule to hybridize to another nucleic acid molecule on the basis of hydrogen bonds between complementary bases. A person skilled in the art knows that two nucleic acid molecules do not need to have 100% complementarity in order to be able to hybridize to one another. Preference is given to a nucleic acid sequence which is to hybridize to another nucleic acid sequence being at least 40%, at least 50%, at least 60%, preferably at least 70%, particularly preferably at least 80%, likewise particularly preferably at least 90%, especially preferably at least 95%, and most preferably at least 98% or 100%, complementary to the latter.

Preference is given to degrees of homology, complementarity and identity to be determined over the entire length of the protein or nucleic acid.

Nucleic acid molecules are identical if they have identical nucleotides in the same 5′-3′ order.

Consequently, the invention also relates to a process for preparing a vector or an expression construct, which process comprises inserting the nucleic acid molecule of the invention into a vector or an expression construct.

Consequently, the invention also relates to a nucleic acid construct or vector comprising the nucleic acid molecule of the invention or prepared in the process of the invention or comprising a nucleic acid construct suitable for use in the process of the invention.

The invention consequently relates to expression constructs comprising, under the genetic control of regulatory nucleic acid sequences, a nucleic acid sequence coding for a polypeptide of the invention; and also to vectors comprising at least one of these expression constructs.

Such constructs of the invention preferably comprise a promoter 5′-upstream of the particular coding sequence and a terminator sequence 3′-downstream and also, if appropriate, further customary regulatory elements which are in each case operatively linked to the coding sequence.

An “operative linkage” means the sequential arrangement of promoter, coding sequence, terminator and, if appropriate, further regulatory elements in such a way that each of the regulatory elements is able to fulfill its function as required in expressing the coding sequence. Examples of operatively linkable sequences are targeting sequences and also enhancers, polyadenylation signals and the like. Other regulatory elements comprise selectable markers, amplification signals, origins of replication and the like. Suitable regulatory sequences are described, for example, in Goeddel, Gene Expression Technology: Methods in Enzymology 185, Academic Press, San Diego, Calif. (1990).

A nucleic acid construct of the invention means in particular those in which the gene for a conversion of the invention has been operatively or functionally linked to one or more regulatory signals for the purpose of regulating, e.g. increasing, expression of the gene.

In addition to these regulatory sequences, the natural regulation of these sequences may still be present upstream of the actual structural genes and, if appropriate, may have been genetically altered in such a way that the natural regulation has been switched off and expression of the genes has been increased. However, the nucleic acid construct may also have a simpler design, i.e. no additional regulatory signals have been inserted upstream of the coding sequence and the natural promoter, together with its regulation, has not been removed. Instead of this, the natural regulatory sequence is mutated in such a way that there is no longer any regulation and expression of the gene is increased.

A preferred nucleic acid construct also advantageously comprises one or more of the previously mentioned enhancer sequences which are functionally linked to the promoter and which enable expression of the nucleic acid sequence to be increased. Additional advantageous sequences such as further regulatory elements or terminators may also be inserted at the 3′ end of the DNA sequences. The nucleic acids of the invention may be present in the construct in one or more copies. The construct may also comprise additional markers such as antibiotic resistances or auxotrophy-complementing genes, if appropriate for the purpose of selecting said construct.

Regulatory sequences which are advantageous for the process of the invention are present, for example, in promoters such as the cos, tac, trp, tet, trp-tet, lpp, lac, lpp-lac, lacI^{q}, T7, T5, T3, gal, trc, ara, rhaP (rhaP_{BAD})SP6, lambda-P_{R} or lambda-P_{L} promoter, which promoters are advantageously used in Gram-negative bacteria. Further advantageous regulatory sequences are present, for example, in the Gram-positive promoters amy and SPO2, in the yeast or fungal promoters ADC1, MFalpha, AC, P-60, CYC1, GAPDH, TEF, rp28, ADH. The pyruvate decarboxylase and methanoloxidase promoters, for example from Hansenula, are also advantageous in this connection. It is also possible to use artificial promoters for regulation.

For the purpose of expression in a host organism, the nucleic acid construct is advantageously inserted into a vector such as a plasmid or a phage, for example, which enables the genes to be expressed optimally in the host. Vectors mean, in addition to plasmids and phages, also any other vectors known to the skilled worker, i.e., for example, viruses such as SV40, CMV, baculovirus and adenovirus, transposons, IS elements, phasmids, cosmids, and linear or circular DNA. These vectors may be replicated autonomously in the host organism or replicated chromosomally. These vectors constitute a further embodiment of the invention. Examples of suitable plasmids are pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, Igt11 or pBdCl, in E. coli, pIJ101, pIJ364, pIJ702 or pIJ361, in Streptomyces, pUB110, pC194 or pBD214, in Bacillus, pSA77 or pAJ667, in Corynebacterium, pALS1, pIL2 or pBB116, in fungi, 2alphaM, pAG-1, YEp6, YEp13 or pEMBLYe23, in yeasts, or pLGV23, pGHlac⁺, pBIN19, pAK2004 or pDH51, in plants. Said plasmids are a small selection of the possible plasmids. Other plasmids are well known to the skilled worker and can be found, for example, in the book Cloning Vectors (Eds. Pouwels P. H. et al. Elsevier, Amsterdam-New York-Oxford, 1985, ISBN 0 444 904018).

For the purpose of expressing the other genes which are present, the nucleic acid construct advantageously also comprises 3′-terminal and/or 5′-terminal regulatory sequences for increasing expression, which are selected for optimal expression in dependence on the host organism and the gene or genes selected.

These regulatory sequences are intended to enable the genes and protein expression to be specifically expressed. Depending on the host organism, this may mean, for example, that the gene is expressed or overexpressed only after induction or that it is expressed and/or overexpressed immediately.

In this connection, the regulatory sequences or factors may preferably influence positively and thereby increase expression of the genes which have been introduced. Thus, the regulatory elements may advantageously be enhanced at the level of transcription by using strong transcription signals such as promoters and/or enhancers. However, in addition to this, it is also possible to enhance translation by improving the stability of the mRNA, for example.

In a further embodiment of the vector, the vector which comprises the nucleic acid construct of the invention or the nucleic acid of the invention may also advantageously be introduced into the microorganisms in the form of a linear DNA and be integrated into the genome of the host organism by way of heterologous or homologous recombination. This linear DNA may consist of a linearized vector such as a plasmid or only of the nucleic acid construct or the nucleic acid of the invention.

In order to be able to express heterologous genes optimally in organisms, it is advantageous to alter the nucleic acid sequences in accordance with the specific codon usage employed in the organism. The codon usage can readily be determined with the aid of computer analyses of other known genes from the organism in question.

An expression cassette of the invention is prepared by fusing a suitable promoter to a suitable coding nucleotide sequence and to a terminator signal or polyadenylation signal. Common recombination and cloning techniques, as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and also in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987) are used for this purpose.

In order to achieve expression in a suitable host organism, the recombinant nucleic acid construct or gene construct is advantageously inserted into a host-specific vector which enables the genes to be expressed optimally in the host. Vectors are well known to the skilled worker and may be found, for example, in “Cloning Vectors” (Pouwels P. H. et al., Eds., Elsevier, Amsterdam-New York-Oxford, 1985).

Consequently, the invention also relates to a host cell which has been transformed or transfected stably or transiently with the vector of the invention or with the polynucleotide of the invention or in which the polynucleotide of the invention or a polynucleotide suitable for the process of the invention is expressed as described above or in which such a polynucleotide is expressed at an increased level compared to a wild type.

It is possible to prepare, with the aid of the vectors or constructs of the invention, recombinant microorganisms which are, for example, transformed with at least one vector of the invention and which may be used for producing the polypeptides of the invention. Advantageously, the above-described recombinant constructs of the invention are introduced into a suitable host system and expressed. In this connection, familiar cloning and transfection methods known to the skilled worker, such as, for example, coprecipitation, protoplast fusion, electroporation, retroviral transfection and the like, are preferably used in order to cause said nucleic acids to be expressed in the particular expression system. Suitable systems are described, for example, in Current Protocols in Molecular Biology, F. Ausubel et al., Eds., Wiley Interscience, New York 1997, or Sambrook et al, Molecular Cloning: A Laboratory Manual. 2nd edition, Cold Spring Harbor Laboratory, Cold Spring Harbor Laboratory Press, Cold Spring Harbor, N.Y., 1989.

According to the invention, it is also possible to prepare homologously recombined microorganisms. For this purpose, a vector which comprises at least one section of a gene of the invention or of a coding sequence in which, if appropriate, at least one amino acid deletion amino acid addition or amino acid substitution has been introduced in order to modify, for example functionally disrupt, the sequence of the invention (knock out vector), is prepared. The introduced sequence may also be a homolog from a related microorganism or be derived from a mammalian, yeast or insect source, for example. Alternatively, the vector used for homologous recombination may be designed in such a way that the endogenous gene is, in the case of homologous recombination, mutated or otherwise altered but still encodes the functional protein (e.g. the upstream regulatory region may have been altered in such a way that expression of the endogenous protein is thereby altered). The altered section of the gene of the invention is in the homologous recombination vector. The construction of vectors which are suitable for homologous recombination is described, for example, in Thomas, K. R. and Capecchi, M. R. (1987) Cell 51:503.

Recombinant host organisms suitable for the nucleic acid of the invention or the nucleic acid construct are in principle any prokaryotic or eukaryotic organisms. Advantageously, microorganisms such as bacteria, fungi or yeasts are used as host organisms. Gram-positive or Gram-negative bacteria, preferably bacteria of the families Enterobacteriaceae, Pseudomonadaceae, Rhizobiaceae, Streptomycetaceae or Nocardiaceae, particularly preferably bacteria of the genera Escherichia, Pseudomonas, Streptomyces, Nocardia, Burkholderia, Salmonella, Agrobacterium or Rhodococcus, are advantageously used. Very particular preference is given to the genus and species Escherichia coli. In addition, further advantageous bacteria can be found in the group of the alpha-proteobacteria, beta-proteobacteria or gamma-proteobacteria.

In this connection, the host organism or host organisms of the invention comprise(s) preferably at least one of the nucleic acid sequences, nucleic acid constructs or vectors which are described in this invention and which encode an enzyme with activity of the invention of converting compound I to give II.

The organisms used in the process of the invention are, depending on the host organism, grown or cultured in a manner known to the skilled worker. Microorganisms are usually grown in a liquid medium which comprises a carbon source, usually in the form of sugars, a nitrogen source, usually in the form of organic nitrogen sources such as yeast extract or salts such as ammonium sulfate, trace elements such as iron salts, manganese salts, magnesium salts and, it appropriate, vitamins, at temperatures of between 0° C. and 100° C., preferably between 10° C. and 60° C., while being gassed with oxygen. In this connection, the pH of the nutrient liquid may or may not be kept at a fixed value, i.e. may or may not be regulated during cultivation. The cultivation may be carried out batchwise, semibatchwise or continuously. Nutrients may be introduced at the beginning of the fermentation or be fed in subsequently in a semicontinuous or continuous manner. The ketone may be added directly to the culture or, advantageously, after cultivation. The enzymes may be isolated from the organisms by using the process described in the examples or be used for the reaction as a crude extract.

The invention furthermore relates to processes for recombinantly preparing polypeptides of the invention or functional, biologically active fragments thereof, with a polypeptide-producing microorganism being cultured, if appropriate expression of the polypeptides being induced and said polypeptides being isolated from the culture. The polypeptides may also be produced in this way on an industrial scale if this is desired.

The recombinant microorganism may be cultured and fermented by known methods. Bacteria may, for example, be propagated in TB medium or LB medium and at a temperature of from 20 to 40° C. and a pH of from 6 to 9. Suitable culturing conditions are described in detail, for example, in T. Maniatis, E. F. Fritsch and J. Sambrook, Molecular Cloning: A Laboratory Manual, Cold Spring Harbor Laboratory, Cold Spring Harbor, N.Y. (1989).

If the polypeptides are not secreted into the culture medium, the cells are then disrupted and the product is obtained from the lysate by known protein isolation processes. The cells may be disrupted, as desired, by means of high-frequency ultrasound, by means of high pressure, such as, for example, in a French pressure cell, by means of osmolysis, by the action of detergents, lytic enzymes or organic solvents, by using homogenizers or by a combination of two or more of the processes listed.

The polypeptides may be purified using known chromatographic methods such as molecular sieve chromatography (gel filtration), for example Q Sepharose chromatography, ion exchange chromatography and hydrophobic chromatography, and also using other customary methods such as ultrafiltration, crystallization, salting-out, dialysis and native gel electrophoresis. Suitable processes are described, for example, in Cooper, F. G., Biochemische Arbeitsmethoden, Verlag Walter de Gruyter, Berlin, N.Y. or in Scopes, R., Protein Purification, Springer Verlag, New York, Heidelberg, Berlin.

It may be advantageous to isolate the recombinant protein by using vector systems or oligonucleotides which extend the cDNA by particular nucleotide sequences and thereby code for altered polypeptides or fusion proteins which are used, for example, to simplify purification. Examples of suitable modifications of this kind are “tags” acting as anchors, such as the modification known as the hexa-histidine anchor, or epitopes which can be recognized as antigens by antibodies (described, for example, in Harlow, E. and Lane, D., 1988, Antibodies: A Laboratory Manual. Cold Spring Harbor (N.Y.) Press). These anchors may be used for attaching the proteins to a solid support such as a polymer matrix, for example, which may, for example, be packed into a chromatography column or may be used on a microtiter plate or on another support.

At the same time, these anchors may also be used for identifying the proteins. The proteins may also be identified by using customary markers such as fluorescent dyes, enzyme markers which, after reaction with a substrate, form a detectable reaction product, or radioactive markers, either on their own or in combination with the anchors, for derivatizing said proteins.

It is also possible to employ in the process of the invention organisms, in particular microorganisms, which have increased acetonitrilase activity or in which the activity of the polypeptide of the invention is at an elevated level compared to the wild type. Such an increase may be achieved, for example, by introducing an appropriate nucleic acid construct such as, for example, the nucleic acid construct or vector of the invention, or by specific or unspecific mutagenesis of the organism. The selected microorganisms are mutagenized according to the invention. Mutagenized means that mutations are introduced specifically or unspecifically into the genetic information, i.e. into the genome of said microorganisms. Specific or unspecific mutations modify one or more pieces of genetic information, i.e. the microorganisms are genetically modified. This modification usually results in faulty or no expression of the affected genes so that the activity of the gene product is reduced or inhibited.

Specific mutations mutate a particular gene or inhibit, reduce or modify its activity. Unspecific mutations mutate randomly one or more genes or inhibit, reduce or modify its/their activity.

In order to carry out specific mutations in a large number of microorganisms, a population may be transformed, for example, with a DNA population or library which is suitable for inhibiting various genes, as many genes as possible, or, in the optimal case, all genes, so that, from a statistical point of view, one, preferably identifiable, DNA fragment is integrated into each gene of the microorganism. The knocked-out gene can be identified by analyzing the site of integration.

In the case of unspecific mutations, a large number of microorganisms is treated with a mutagenic reagent. The amount of reagent or intensity of treatment is chosen so that, from a statistical point of view, one mutation per gene takes place. Methods and reagents for the mutagenesis of microorganisms are sufficiently known to the skilled worker. The practical implementation of the various methods can be found in numerous publications, for example also in A. M. van Harten (1998) “Mutation breeding: theory and practical applications”, Cambridge University Press, Cambridge, UK, E Friedberg, G Walker, W Siede (1995), “DNA Repair and Mutagenesis”, Blackwell Publishing, K. Sankaranarayanan, J. M. Gentile, L. R. Ferguson (2000) “Protocols in Mutagenesis”, Elsevier Health Sciences. A person skilled in the art knows that the rate of spontaneous mutation in cells is very low and that there are a large number of chemical, physical and biological agents which can induce mutations. These agents are referred to as mutagens. A distinction is made between biological, physical and chemical mutagens.

There are various classes of chemical mutagens which differ in their mode of action: for example, base analogs such as, for example 5-bromouracil, 2-aminopurine; chemicals reacting with DNA, such as, for example, nitrous acid, hydroxylamine; or alkylating compounds such as monofunctional (e.g. ethyl methanesulfonate, dimethyl sulfate, methyl methanesulfonate), bifunctional (e.g. nitrogen mustard gas, mitomycin, nitrosoguanidines- dialkylnitrosamines, N-nitrosourea derivatives, N-alkyl-N-nitro-N-nitrosoguanidines-), intercalating dyes (e.g. acridines, ethidium bromide). Physical mutagenization is carried out, for example, by way of irradiation of the organisms. Several forms of irradiation are strong mutagens. Two classes can be distinguished: non-ionizing radiation (e.g. UV) and ionizing radiation (e.g. X radiation), Mutations may also be induced by biological processes. The standard procedure here is transposon mutagenesis which results in the modification, usually the loss, of a gene activity, due to insertion of a transposable element within or in the vicinity of a gene. By identifying the site of insertion of the transposon, the gene whose activity has been altered may be isolated.

Mutagenesis may alter the cellular activity of one or more gene products. The cellular activity of the arylacetonitrilase described herein, particularly preferably of the polypeptide described herein, is preferably increased.

Preferably, it is possible to prepare the organisms which are non-transgenic according to the invention, in particular microorganisms, plants and plant cells which are distinguished by a modulation of the expression and/or the binding behavior of the endogenous arylacetonitrilase and which have a permanent or transient resistance to pathogens, by the “TILLING” approach (Targeting Induced Local Lesion in Genomes). This method has been described in detail in Colbert et al. (2001, Plant Physiology, 126, 480-484), McCallum et al. (2000, Nat. Biotechnol., 18, 455-457) and McCallum et al. (2000, Plant Physiology, 123, 439-442). The abovementioned references are incorporated herein explicitly as disclosure with respect to the “TILLING” method.

The TILLING method is a strategy of “reverse genetics”, which combines the production of high densities of point mutations in mutagenized collections of microorganisms or plants, for example by chemical mutagenesis with ethyl methanesulfonate (EMS), with the rapid systematic identification of mutations in target sequences. The target sequence is first amplified by PCR into DNA pools of mutagenized M2 populations. Denaturation and annealing reactions of the heteroallelic PCR products allow the formation of heteroduplexes in which one DNA strand is from the mutated and the other one from the wild-type PCR product. At the site of the point mutation, a “mismatch” occurs which can be identified either via denaturing HPLC (DHPLC, McCallum et al., 2000, Plant Physiol., 123, 439-442) or by the CelI mismatch detection system (Oleykowsky et al., 1998, Nucl. Acids Res. 26, 4597-4602). CelI is an endonuclease which recognizes mismatches in heteroduplex DNA and specifically cleaves said DNA at these sites. The cleavage products can then be fractionated and detected via automated sequencing gel electrophoresis (Colbert et al., 2001, vide supra). After identification of target gene-specific mutations in a pool, individual DNA samples are appropriately analyzed in order to isolate the microorganism or the plant containing the mutation. In this way, in the case of the microorganisms, plants and plant cells of the invention, the mutagenized plant cells or plants are identified, after the mutagenized populations have been produced using primer sequences specific for arylacetonitrilase. The TILLING method is generally applicable to any microorganisms and plants and plant cells.

In one embodiment, the invention also relates to a composition comprising essentially R- and/or S-5-norbornene-2-endo-carbonitrile and to compositions comprising more than 60%, 70%, 80%, 90%, 95%, 99% of R- and/or S-5-norbornene-2-endo-carboxylic acid; and/or comprising an R- and/or S-5-norbornene-2-exo-carboxylic acid ratio of less than 40%, 30%, 20%, 10%, 5%, 1%. Such a composition has not been prepared previously in the prior art. Chemical preparation of norbornene acid always resulted in a mixture of enantiomers of a 5-norbornene-2-endo-carboxylic acid to 5-norbornene-2-exo-carboxylic acid ratio of approximately 0.6:approximately 0.4.

The present invention also relates to a composition comprising essentially R- and/or S-5-norbornene-2-exo-carbonitrile and to a composition comprising R- and/or S-5-norbornene-2-endo-carboxylic acid to R- and/or S-5-norbornene-2-exo-carboxylic acid in a ratio of less than 0.6 to greater than 0.4. Such a composition has not been prepared previously in the prior art. Chemical preparation of norbornene acid always resulted in a mixture of enantiomers of a 5-norbornene-2-endo-carboxylic acid to 5-norbornene-2-exo-carboxylic acid ratio of approximately 0.6:approximately 0.4.

Consequently, the invention also relates to a composition which can be prepared according to the process of the invention. In one embodiment, the invention relates to a composition prepared according to the process of the invention.

In a further embodiment, the invention relates to the use of an enzyme, in particular of a nitrilase, preferably of an arylacetonitrilase, particularly preferably of a polypeptide of the invention having the sequence depicted in SEQ ID NO: 2 or 4, or a homolog or a functional fragment thereof for enriching one isomer of the compound II from a mixture of isomers of compound I.

In a further embodiment, the invention relates to the use of an enzyme, in particular of a nitrilase, preferably of an arylacetonitrilase, particularly preferably of a polypeptide of the invention having the sequence depicted in SEQ ID NO: 2 or 4, or a homolog or a functional fragment thereof for enriching R- and/or S-5-norbornene-2-endo-carboxylic acid from a mixture comprising R- and/or S-5-norbornene-2-endo-carbonitrile and R- and/or S-5-norbornene-2-exo-carbonitrile.

The invention furthermore relates to the use of an arylacetonitrilase for converting R- and/or S-5-norbornene-2-endo-carbonitrile and/or R- and/or S-5-norbornene-2-exo-carbonitrile to give R- and/or S-norbornene-2-endo-carboxylic acid and/or R- and/or S-norbornene-2-exo-carboxylic acid.

The invention moreover relates to the use of an arylacetonitrilase for converting R- and/or S-5-norbornene-2-endo-carbonitrile and/or R- and/or S-5-norbornene-2-exo-carbonitrile to give R- and/or S-endo- and/or R- and/or S-norbornene-2-exo-carboxylic acid.

The invention moreover relates to the use of an enzyme, in particular of a nitrilase, preferably of an arylacetonitrilase, particularly preferably of a polypeptide of the invention having the sequence depicted in SEQ ID NO: 2 or 4, or a homolog or a functional fragment thereof for converting R- and/or S-5-norbornene-2-endo-carbonitrile to give the isomerically pure R- and/or S-5-norbornene-2-endo-carboxylic acid with a high substrate concentration.

In a further embodiment, the invention relates to the use of an enzyme, in particular of a nitrilase, preferably of an arylacetonitrilase, particularly preferably of a polypeptide of the invention, wherein a polypeptide is used which is encoded by a nucleic acid molecule comprising a nucleic acid molecule selected from the group consisting of:
(a) nucleic acid molecule which encodes a polypeptide depicted in SEQ ID NO: 2 or 4;
(b) nucleic acid molecule which comprises at least the polynucleotide of the coding sequence according to SEQ ID NO: 1 or 3;
(c) nucleic acid molecule whose sequence, owing to the degeneracy of the genetic code, may be derived from a polypeptide sequence encoded by a nucleic acid molecule according to (a) or (b);
(d) nucleic acid molecule which encodes a polypeptide whose sequence is at least 60% identical to the amino acid sequence of the polypeptide encoded by the nucleic acid molecule according to (a) or (b);
(e) nucleic acid molecule which encodes a polypeptide derived from an arylaceto-nitrilase polypeptide in which up to 25% of the amino acid residues have been modified by deletion, insertion, substitution or a combination thereof compared to SEQ ID NO: 2 or 4 and which still retains at least 30% of the enzymatic activity of SEQ ID NO: 2 or 4; and
(f) nucleic acid molecule which encodes a fragment or an epitope of an arylacetonitrilase encoded by any of the nucleic acid molecules according to (a) to (c);
   or comprising a complementary sequence thereof.

In one embodiment, the polypeptide does not have the sequence according to SEQ ID NO: 2 or 4. In one embodiment, the polypeptide neither has the sequence of the nitrilase mentioned in Eur. J. Biochem. 182, 349-156, 1989. In one embodiment, the polypeptide neither has the sequence of the database entry AY885240.

Finally, the invention relates to the use of a polypeptide for preparing a compound of the formula II by enzymatically converting a compound of the formula I wherein the polypeptide is encoded by a nucleic acid molecule comprising a nucleic acid molecule selected from the group consisting of:
(a) nucleic acid molecule which encodes a polypeptide depicted in SEQ ID NO: 2 or 4;
(b) nucleic acid molecule which comprises at least the polynucleotide of the coding sequence according to SEQ ID NO: 1 or 3;
(c) nucleic acid molecule whose sequence, owing to the degeneracy of the genetic code, may be derived from a polypeptide sequence encoded by a nucleic acid molecule according to (a) or (b);
(d) nucleic acid molecule which encodes a polypeptide whose sequence is at least 60% identical to the amino acid sequence of the polypeptide encoded by the nucleic acid molecule according to (a) or (b);
(e) nucleic acid molecule which encodes a polypeptide derived from an arylaceto-nitrilase polypeptide in which up to 25% of the amino acid residues have been modified by deletion, insertion, substitution or a combination thereof compared to SEQ ID NO: 2 or 4 and which still retains at least 30% of the enzymatic activity of SEQ ID NO: 2 or 4; and
(f) nucleic acid molecule which encodes a fragment or an epitope of an arylacetonitrilase encoded by any of the nucleic acid molecules according to (a) to (c);
   or comprising a complementary sequence thereof.

In one embodiment, the polypeptide does not have the sequence according to SEQ ID NO: 2 or 4. In one embodiment, the polypeptide neither has the sequence of the nitrilase mentioned in Eur. J. Biochem. 182, 349-155, 1989. In one embodiment, the polypeptide neither has the sequence of the database entry AY885240.

### FIGURES

FIG. 1 depicts enzymes having activity of the invention. When using the isomerically pure exo-norbornene nitrile, a high activity was observed. A high activity was also observed at a high nitrite concentration.

The above description and the examples below serve only to illustrate the invention. The numerous possible modifications which are obvious to the skilled worker are likewise comprised according to the invention.

### EXAMPLES

### 1. Conversion of 5-norbornene-2-endo/exo-carbonitrile with Various Nitrilases

Nitrilases from Biocatalytics (“Nit101-108”) were used as BTM at 2 mg/ml. The BASF nitrilases were used as recombinant whole-cell biocatalysts (E. coli TG10pDHE system with GroELS chaperones, cf. PCT/EP 03113367) and were grown for this purpose in 30 ml of LB containing ampicillin (100 μg/ml), spectinomycin (100 μg/ml), chloramphenicol (20 μg/ml), IPTG (0.1 mM) and rhamnose monohydrate (0.5 g/L) in a 100-ml Erlenmeyer flask at 37° C. overnight. The cells were washed 1× in 30 ml of 10 mM Pipes, pH 7.0, and taken up in 3 ml of buffer and, if appropriate, stored at −20° C. The nitrile used was the mixture of isomers from Aldrich.

### Assay:

10-200 μl of cells (10 times concentrated)
100 μl, 100 mM of nitrile in MeOH
ad 1000 μl, with 10 mM Pipes pH 7.0
3 to 21 h of shaking at 40° C.

The samples were centrifuged and the supernatants were assayed for 5-norbornene-2-endo/exo-carboxylic acid via RP-HPLC.

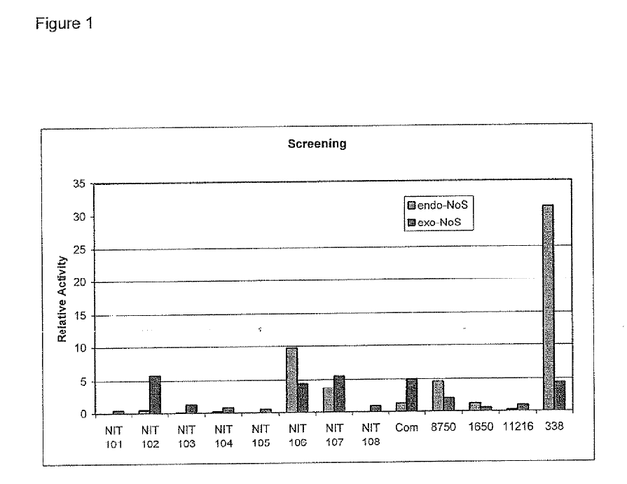

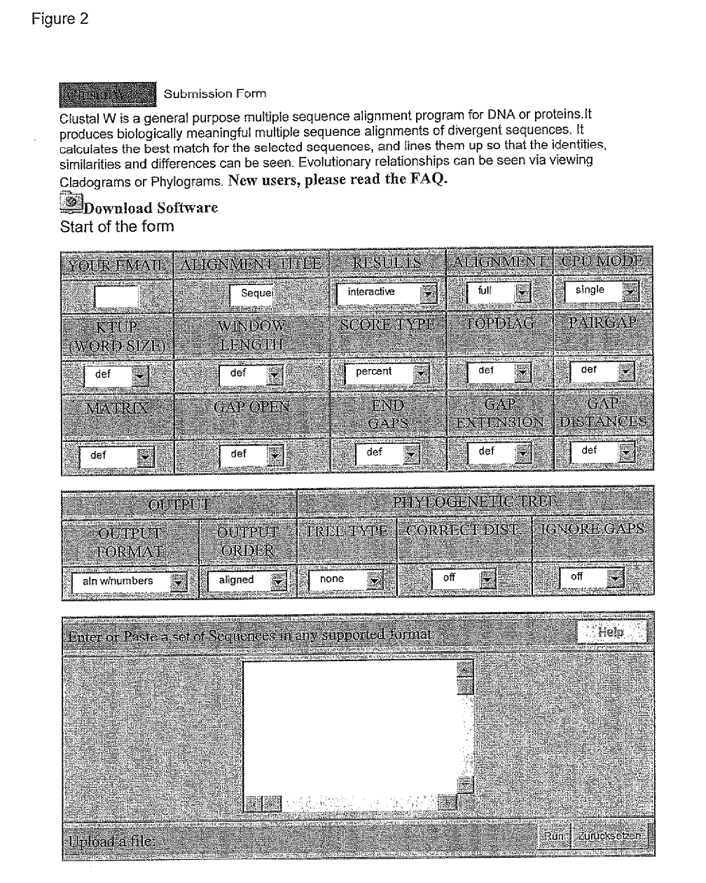

The results are depicted in the diagram of FIG. 1.

### 2. Conversion of 5-norbornene-2-endo-carbonitrile with Nitrilase 338 and Isolation

30 ml of nitrile and 1-20 g/L TG10+pDHE338 cells were stirred in 0.5 L of 10 mM NaH2PO4, pH 7.5 in a glass reactor at 250 rpm and 40° C. After 7-24 h, conversion to 5-norbornene-2-endo-carboxylic acid was analyzed via HPLC and turned out to be almost complete (<3 mM nitrile).

After the cells had been removed, crude 5-norbornene-2-carboxylic acid was concentrated in a rotary evaporator (approx. 2 M) and extracted with one volume of heptane under acidic conditions (pH 2 with H₂SO₄). After evaporation of the solvent and drying, 5-norbornene-2-endo-carboxylic acid was obtained as solids (mp. 46° C.) in greater than 99% purity (H-NMR, HPLC).

### 3. Conversion of 5-norbornene-2-exo-carbonitrile with Nitrilase 338 and Isolation

30 ml of nitrile and 1-20 g/L TG10+pDHE338 cells were stirred in 0.5 L of 10 mM NaH2PO4, pH 7.5 in a glass reactor at 250 rpm and 40° C. After 1-7 d, conversion to 5-norbornene-2-endo-carboxylic acid was analyzed via HPLC and turned out to be almost complete (<3 mM nitrile).

After the cells had been removed, crude 5-norbornene-2-carboxylic acid was concentrated in a rotary evaporator (approx. 2 M) and extracted with one volume of heptane under acidic conditions (pH 2 with H₂SO₄). After evaporation of the solvent and drying, 5-norbornene-2-endo-carboxylic acid was obtained as solids (mp. 42° C.) in greater than 99% purity (H-NMR, HPLC).

### 4. Comparative Example Rhodococcus rhodochrous J1-Nitrilase, Cloning and Expression

In order to clone the nitrilase of Rhodococcus rhodochrous J1 (FERM BP-1478), the primers Mke638 and Mke639 were selected on the basis of the sequence D11425 (J. Biol. Chem. 207 (29), 20740-20751 (1992)), and the nitrilase gene was amplified from a single colony of the strain by means of PCR.

### PCR:

| | | Gene |
|---|---|---|
| Template | Primer | length |
| Colony of R. rhodochrous J1 | Mke638 + Mke639 | 1191 bp |

### Primers:

| Primer No. | Sequence (5′-3′) | Position |
|---|---|---|
| Mke638 | CCCAAGCTTACGATCGACGATGCGTTG | C-terminal |
| | (SEQ ID NO: 5) | primer |
| | | (HindIII) |
| Mke639 | GGGAATTCCATATGGTCGAATACACAA | N-terminal |
| | ACAC | primer |
| | (SEQ ID NO: 6) | (NdeI) |

The PCR was carried out according to the Stratagene standard protocol using Pfu ultrapolymerase (Stratagene) and the following temperature program: 95° C. for 5 minutes; 30 cycles at 95° C. for 45 s, 50° C. for 45 s and 72° C. for 1 min 30 s; 72° C. for 10 min; 10° C. until use. The PCR product (1.2 kb) was isolated via agarose gel electrophoresis (1.2% E-Gel, Invitrogen) and column chromatography (GFX kit, Amersham) and subsequently digested with NdeI/HindIII and cloned into the correspondingly digested pDHE19.2 vector (a pJOE derivative, DE19848129). The ligation mixtures were transformed into E. coli TG10 pAgro4 pHSG575 (TG10: an RhaA⁻ derivative of E. coli TG1 (Stratagene); pAgro4: Takeshita, S; Sato, M; Toba, M; Masahashi, W; Hashimoto-Gotoh, T (1987) Gene 61, 63-74; pHSG575: T. Tomoyasu et al (2001), Mol. Microbiol. 40(2), 397-413). 6 transformants were picked and analyzed: the 6 transformants were grown in 30 mL of LBAmp/Spec/Cm 0.1 mM IPTG/0.5 g/L rhamnose in a 100 mL Erlenmeyer flask (baffles) at 37° C. for 18 h, centrifuged at 5000 g/10 min, washed once with 10 mM KH2PO4 pH 8.0, and resuspended in 3 ml of the same buffer. They were diluted 1:10 with 10 mM KH2PO4 pH 8.0 and 6 mM benzonitrile and assayed for their activity. The samples were centrifuged and the supernatants were assayed for benzoic acid and benzonitrile via RP-HPLC. 4 clones were active and exhibited complete conversion to benzoic acid already after 15 min. Sequencing of these 4 clones revealed that the insert of the plasmid obtained, pDHErrhJ1, was the nucleic acid sequence of R. rhodochrous J1 nitrilase, and depicted in D11245.

### 5. Conversion of 5-norbornene-2-endo/exo-carbonitrile with Various Nitrilases

Rhodococcus rhodochrous J1 (FERM BP-1478) was grown as described in the literature (Nagasawa et al., Arch. Microbiol. 1988; 150, 89-94) and harvested. The cells were assayed for their benzonitrilase activity, as in example 4, and exhibited complete conversion after 15 min. The BASF nitrilase strains and E. coli TG10+pDHE9632J1 (example 4) were grown and harvested as in example 1. Subsequently, the dry biomasses were determined (R. rhodochrous J1: 3.5 g/L, E. coli strains: 0.8 g/L).

### Assay:

×μl of cell suspension (6 g/L BTM)
200-1000 mM of nitrile
0-0.5 mM DTT
ad 1000 μl, with 20 mM KH2PO4 pH 8.0
shaking at 40° C. for 0.3-6 d

In order to monitor the conversion, samples were taken, centrifuged, and the supernatants were assayed for 5-norbornene-2-endo/exo-carboxylic acid and their acid amides via RP-HPLC.

eNOS Formed at Various eNon Concentrations:

| | eNON/ | | | |
|---|---|---|---|---|
| | mM | eNON/mM | eNON/mM | eNON/mM |
| Strain | 200 | 500 | 1000 | 1000/+DTT |
| TG10 + pDHE- | 0.0 | 0.0 | 0.0 | 0.0 |
| 11216 | | | | |
| TG10 + pDHE-338 | 184.9 | 457.3 | 703.8 | 651.4 |
| R. rhodochrous J1 | 0.0 | 0.0 | 0.0 | 0.0 |
| TG10 + pDHE-J1 | 2.0 | — | 0.0 | 0.0 |

eNOSamide Formed at Various eNon Concentrations:

| | eNON/ | | | |
|---|---|---|---|---|
| | mM | eNON/mM | eNON/mM | eNON/mM |
| Strain | 200 | 500 | 1000 | 1000/+DTT |
| TG10 + pDHE- | 0.0 | 0.0 | 0.0 | 0.0 |
| 11216 | | | | |
| TG10 + pDHE-338 | 0.0 | 0.0 | 1.1 | 0.9 |
| R. rhodochrous J1 | 22.1 | 30.2 | 30.8 | 33.5 |
| TG10 + pDHE-J1 | 0.0 | — | 0.0 | 0.0 |

xnOS Formed at Various eNon Concentrations:

| | eNON/ | | | |
|---|---|---|---|---|
| | mM | eNON/mM | eNON/mM | eNON/mM |
| Strain | 200 | 500 | 1000 | 1000/+DTT |
| TG10 + pDHE- | 13.5 | 8.2 | 6.1 | 5.2 |
| 11216 | | | | |
| TG10 + pDHE-338 | 204.5 | 431.8 | 500.3 | 490.2 |
| R. rhodochrous J1 | 0.0 | 0.0 | 0.0 | 0.2 |
| TG10 + pDHE-J1 | 16.0 | — | 9.8 | — |

xNOSamide formed at various eNON concentrations:

| | eNON/ | | | |
|---|---|---|---|---|
| | mM | eNON/mM | eNON/mM | eNON/mM |
| Strain | 200 | 500 | 1000 | 1000/+DTT |
| TG10 + pDHE- | 0.0 | 0.0 | 0.0 | 0.0 |
| 11216 | | | | |
| TG10 + pDHE-338 | 0.0 | 0.0 | 0.0 | 0.0 |
| R. rhodochrous J1 | 49.1 | 24.7 | 28.7 | 50.5 |
| TG10 + pDHE-J1 | 0.0 | — | 0.0 | — |

### Overview of Comparative Sequences:

1. a) Polypeptide sequence of NA nitrilase of Pseudomonas fluorescens EBC191 (DSM7155) from AY885240
2. Polypeptide sequence of Nit nitrilase of ADI64602 (WO2003097810-A2 Seq. ID175)
3. Polypeptide sequence of Nit nitrilase of ADG93882 (WO2003097810-A2 Seq. ID349)

1-27. (canceled)

28. A process for preparing 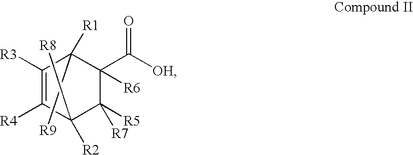
wherein R1-R9 independently are H; linear or branched alkyl having from one to six carbons, cycloalkyl having up to six carbons, unsubstituted aryl having from 3 to 10 carbons, amino-substituted aryl having from 3 to 10 carbons, hydroxy-substituted aryl having from 3 to 10 carbons, or halo-substituted aryl having from 3 to 10 carbons; and
optionally R5 and R7, or R8 and R9, form a cycloalkyl having from 3 to 6 carbons; and optionally R8 and R9, or R5 and R7, carry exocyclic double bonds with optional substituents; and optionally R3 and R4 form a ring (4,5,6) or are part of an annealed aromatic compound,
comprising enzymatically preparing Compound II from 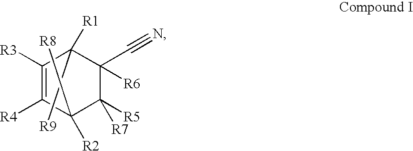
wherein R1 to R9 are as above.

29. The process of claim 28, further comprising the presence of an arylacetonitrilase.

30. The process of claim 28, wherein the enzymatic preparation of compound I comprises incubation with a polypeptide or a medium comprising a polypeptide, and wherein the polypeptide is encoded by a nucleic acid molecule comprising a nucleic acid molecule selected from the group consisting of:
(a) a nucleic acid molecule encoding a polypeptide of SEQ ID NOs: 2 or 4;
(b) a nucleic acid molecule comprising the coding sequence of a polynucleotide of SEQ ID NOs: 1 or 3;
(c) a nucleic acid molecule whose degenerate sequence is derived from a polypeptide sequence encoded by a nucleic acid molecule according to (a) or (b);
(d) a nucleic acid molecule which encodes a polypeptide whose sequence is at least 60% identical to the amino acid sequence of the polypeptide encoded by the nucleic acid molecule according to (a) or (b);
(e) a nucleic acid molecule encoding a polypeptide derived from an arylacetonitrilase polypeptide in which up to 25% of the amino acid residues have been modified by deletion, insertion, substitution or a combination thereof compared to SEQ ID NO: 2, and which retains at least 30% of the enzymatic activity of SEQ ID NO: 2; and
(f) a nucleic acid molecule encoding a fragment or an epitope of an arylacetonitrilase encoded by any of the nucleic acid molecules of (a) to (c);
or comprising a complementary sequence thereof; and, optionally,
wherein the product formed is isolated.

31. The process of claim 29, wherein compound I is selected from the group consisting of R-5-norbornene-2-endo-carbonitrile, S-5-norbornene-2-endo-carbonitrile, R-5-norbornene-2-exo-carbonitrile, and S-5-norbornene-2-exo-carbonitrile.

32. The process of claim 29, wherein compound I is R,S-5-norbornene-2-endo-carbonitrile or R,S-5-norbornene-2-exo-carbonitrile.

33. The process of claim 31, wherein compound I is R-5-norbornene-2-endo-carbonitrile, S-5-norbornene-2-endo-carbonitrile, R-5-norbornene-2-exo-carbonitrile, or S-5-norbornene-2-exo-carbonitrile are hydrolyzed to yield S-5-norbornene-2-exo-carboxylic acid, S-5-norbornene-2-endo-carboxylic acid, R-5-norbornene-2-exo-carboxylic acid or R-5-norbornene-2-endo-carboxylic acid.

34. The process of claim 29, wherein Compound I is an essentially enantiomerically pure substrate.

35. The process of claim 29, wherein the concentration of Compound I is at least 20 mM and 50% or more of Compound I is converted to Compound II.

36. The process of claim 29, wherein Compound I is a mixture of isomers and Compound II is enriched in one isomer.

37. A polypeptide which is encoded by a nucleic acid molecule comprising a nucleic acid molecule selected from the group consisting of:
(a) a nucleic acid molecule encoding the polypeptide of SEQ ID NO: 2;
(b) a nucleic acid molecule comprising the coding sequence of the polynucleotide of SEQ ID NO: 1;
(c) a nucleic acid molecule whose degenerate sequence is derived from a polypeptide sequence encoded by a nucleic acid molecule of (a) or (b);
(d) a nucleic acid molecule encoding a polypeptide whose sequence is at least 60% identical to the amino acid sequence of the polypeptide encoded by the nucleic acid molecule of (a) or (b);
(e) a nucleic acid molecule encoding a polypeptide derived from an arylacetonitrilase polypeptide in which up to 15% of the amino acid residues have been modified by deletion, insertion, substitution or a combination thereof compared to SEQ ID NO: 2, and which retains at least 30% of the enzymatic activity of SEQ ID NO: 2; and
(f) a nucleic acid molecule which encodes a fragment or an epitope of an arylacetonitrilase encoded by any of the nucleic acid molecules of (a) to (c);
or comprising a complementary sequence thereof.

38. The polypeptide of claim 37, which is an arylacetonitrilase.

39. The polypeptide of claim 37, which hydrolyzes 50% or more of Compound I in a composition comprising a 5-norbornene-2-endo-carbonitrile concentration of 200 mM or more.

40. The polypeptide of claim 37, which hydrolyzes 50% or more of Compound I in a composition comprising a 5-norbornene-2-exo-carbonitrile concentration of 200 mM or more.

41. A nucleic acid molecule comprising a polynucleotide encoding the polypeptide of claim 38, wherein the nucleic acid molecule does not have the sequence of SEQ ID NO: 1 or 3.

42. A vector or expression construct comprising the nucleic acid molecule of claim 41.

43. The vector of claim 42, wherein the nucleic acid molecule is functionally linked to a regulatory sequence that allows expression in a prokaryotic or eukaryotic host cell.

44. The host cell of claim 42 that has been transformed, or stably or transiently transfected, with the vector of claim 42 or the nucleic acid molecule of claim 41, or which expresses the nucleic acid molecule of claim 41 or the polypeptide of claim 37.

44. A composition comprising 5-norbornene-2-endo-carbonitrile and an endo-norbornene acid to exo-norbornene acid ratio of ≧0.6:≦0.4.

45. A composition comprising 5-norbornene-2-exo-carbonitrile and an endow norbornene acid to exo-norbornene acid ratio of <0.6:>0.4.

46. A composition prepared by the process of claim 29.


SEQUENCE LISTING (6 sequences)

SEQ ID NO: 1 - DNA, 1056 bp, Rhodococcus rhodochrous
atgacggtgcataaaaaacagtacaaagtagccgcggtgcaggccgcccctgcgttcctcgacctggaagctggcgtggccaaagccatcggactgattgctcaggcggcggctgagggtgcctcactggtcgctttccccgaagcgtggctgccggggtatccctggtggatctggctggactccccggccggcggcatgcgcttcgtccagcgcaacttcgacaatgctctggaggtcggcagcgaacccttcgagcggctctgcagggctgcggcacagcacaaaatctacgtcgtactgggcttcactgaacgctctggcggcaccttgtatttggctcaggcgatcattgatgattgcggtcgggtagtcgccacacggcgtaagctcaagccgactcacgtggagcgctcagtctacggagaaggcgacggtagtgaccttgctgtgcatgacactaccttgggtcgcttaggtgccttgtgctgcgcggagcatatccagccgctgtccaagtacgccatgtacgctcagcacgaacaggtacatatcgcggcctggcctagcttttcggtataccggggggctgcgtttcaactgagcgcccaagccaataatgccgcctcgcaagtctacgcactggaaggtcagtgttttgtgctggcgccatgcgccacggtgtccaaagaaatgctcgacgaactgattgattctccggccaaggctgagctgctgctggaaggtggcggcttcgcgatgatctacggcccggatggcgcaccgctgtgtacgccattggcggaaacagaggagggcattctctatgcggatatcgacttgggggtgatcggggtggccaaagctgcctacgacccggttggtcactattcacgccctgatgtgctgcggttgctggtcaaccgggagccaatgacgcgtgtgcattatgttcagccgcagtcgttaccggagacatcggtgttggcgttcggtgcgggagcggatgccatcagaagtgaggagaacccagaagagcaaggcgacaagggatcc

SEQ ID NO: 2 - protein, 352 aa, Rhodococcus rhodochrous
MTVHKKQYKVAAVQAAPAFLDLEAGVAKAIGLIAQAAAEGASLVAFPEAWLPGYPWWIWLDSPAGGMRFVQRNFDNALEVGSEPFERLCRAAAQHKIYVVLGFTERSGGTLYLAQAIIDDCGRVVATRRKLKPTHVERSVYGEGDGSDLAVHDTTLGRLGALCCAEHIQPLSKYAMYAQHEQVHIAAWPSFSVYRGAAFQLSAQANNAASQVYALEGQCFVLAPCATVSKEMLDELIDSPAKAELLLEGGGFAMIYGPDGAPLCTPLAETEEGILYADIDLGVIGVAKAAYDPVGHYSRPDVLRLLVNREPMTRVHYVQPQSLPETSVLAFGAGADAIRSEENPEEQGDKGS

SEQ ID NO: 3 - DNA, 1053 bp, Rhodococcus rhodochrous
atgacggtgcataaaaaacagtacaaagtagccgcggtgcaggccgcccctgcgttcctcgacctggaagctggcgtggccaaagccatcggactgattgctcaggcggcggctgagggtgcctcactggtcgctttccccgaagcgtggctgccggggtatccctggtggatctggctggactccccggccggcggcatgcgcttcgtccagcgcaacttcgacaatgctctggaggtcggcagcgaacccttcgagcggctctgcagggctgcggcacagcacaaaatctacgtcgtactgggcttcactgaacgctctggcggcaccttgtatttggctcaggcgatcattgatgattgcggtcgggtagtcgccacacggcgtaagctcaagccgactcacgtggagcgctcagtctacggagaaggcgacggtagtgaccttgctgtgcatgacactaccttgggtcgcttaggtgccttgtgctgcgcggagcatatccagccgctgtccaagtacgccatgtacgctcagcacgaacaggtacatatcgcggcctggcctagcttttcggtataccggggggctgcgtttcaactgagcgcccaagccaataatgccgcctcgcaagtctacgcactggaaggtcagtgttttgtgctggcgccatgcgcaccggtgtccaaagaaatgctcgacgaactgattgattctccggccaaggctgagctgctgctggaaggtggcggcttcgcgatgatctacggcccggatggcgcaccgctgtgtacgccattggcggaaacagaggagggcattctctatgcggatatcgacttgggggtgatcggggtggccaaagctgcctacgacccggttggtcactattcacgccctgatgtgctgcggttgctggtcaaccgggagccaatgacgcgtgtgcattatgttcagccgcagtcgttaccggagacatcggtgttggcgttcggtgcgggagcggatgccatcagaagtgaggagaacccagaagagcaaggcgacaagtag

SEQ ID NO: 4 - protein, 350 aa, Rhodococcus rhodochrous
MTVHKKQYKVAAVQAAPAFLDLEAGVAKAIGLIAQAAAEGASLVAFPEAWLPGYPWWIWLDSPAGGMRFVQRNFDNALEVGSEPFERLCRAAAQHKIYVVLGFTERSGGTLYLAQAIIDDCGRVVATRRKLKPTHVERSVYGEGDGSDLAVHDTTLGRLGALCCAEHIQPLSKYAMYAQHEQVHIAAWPSFSVYRGAAFQLSAQANNAASQVYALEGQCFVLAPCAPVSKEMLDELIDSPAKAELLLEGGGFAMIYGPDGAPLCTPLAETEEGILYADIDLGVIGVAKAAYDPVGHYSRPDVLRLLVNREPMTRVHYVQPQSLPETSVLAFGAGADAIRSEENPEEQGDK

SEQ ID NO: 5 - DNA, 27 bp, Artificial Sequence
  Synthetic Primer
cccaagcttacgatcgacgatgcgttg

SEQ ID NO: 6 - DNA, 31 bp, Artificial Sequence
  Synthetic Primer
gggaattccatatggtcgaatacacaaacac